(19) United States
(12) Patent Application Publication
(10) Pub. No.: US 2004/0120848 A1
(43) Pub. Date: Jun. 24, 2004

(54) Method for manufacturing a sterilized and calibrated biosensor-based medical device

(76) Inventor: Maria Teodorczyk, San Jose, CA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/324,933

(22) Filed: Dec. 20, 2002

(51) Int. Cl. A61L 2/08

(52) U.S. Cl. 422/22; 436/8

(57) ABSTRACT

A method for manufacturing a sterilized and calibrated biosensor-based medical device (e.g., an integrated biosensor and lancet medical device) includes sterilizing a biosensor-based medical device that contains a biosensor reagent composition (e.g., an analyte specific enzyme and mediator biosensor reagent composition). The sterilizing can be accomplished using, for example, a gamma radiation based technique. Thereafter, the biosensor reagent composition of the sterilized biosensor-based medical device is calibrated. Another method for manufacturing a sterilized and calibrated biosensor-based medical device includes first assembling and packaging a plurality of biosensor-based medical devices that include a biosensor reagent composition. The packaged biosensor-based medical devices are then sterilized using a radiation-based sterilization technique, to create a plurality of sterilized, packaged biosensor-based medical devices. Thereafter, the sterilized and packaged biosensor-based medical devices are calibrated. The calibration can be accomplished, for example, using a statistical sample of the plurality of sterilized, packaged biosensor-based medical devices.

### BACKGROUND OF THE INVENTION

1. Field of the Invention

This invention relates, in general, to methods for the manufacturing of medical devices and, in particular, to methods for manufacturing sterilized and calibrated medical devices.

2. Description of the Related Art

Radiation-based sterilization of specific types of medical devices is common and widespread today due to both favorable economics and reliability. Depending on the type of medical device to be sterilized, radiation-based sterilization can be accomplished using either electromagnetic or particle radiation. Ionizing radiation in the electromagnetic spectrum (e.g., gamma [γ], x-ray and electron radiation) can produce bactericidal effects by transferring photon energy into characteristic ionizations in or near a biological target (e.g., detrimental microorganisms). In addition to the pairs of positive and negative ions that are created by such characteristic ionizations, free radicals and activated molecules can also be produced in medical devices undergoing radiation-based sterilization.

Gamma radiation has been commonly used to sterilize non-bioactive medical devices, including common hospital supplies such as plastic hypodermic syringes and sutures. Gamma radiation can successfully destroy detrimental microorganisms without increasing the temperature of the medical device undergoing radiation-based sterilization. Therefore, radiation-based sterilization that utilizes gamma radiation is often referred to as “cold sterilization.” A minimum standard dose of 25 kGy of radiation has been routinely used in medical device sterilization. This dose can provide a safety factor equivalent to 10⁻⁶ inactivation of the most resistant microorganisms.

Exposure to radiation-induced energy can alter chemicals, including water, by prompting their ionization, decomposition and the production of free radicals. In the presence of oxygen, such free radicals can form hydrogen peroxide and/or hydroperoxyl radicals that act as oxidizing or reducing agents. These agents can subsequently degrade and otherwise alter a variety of chemicals and biochemicals (e.g., enzymes).

Gamma sterilization could be considered appropriate for complete destruction of microbial flora in biosensor-based medical devices (e.g., disposable glucose sensors which combine lancing, sample transfer and glucose concentration measuring components in a single integral medical device). However, sterilization of biosensor-based medical devices containing analyte specific reagents (i.e., biosensor reagent compositions such as analyte specific enzymes and associated mediators) has not heretofore been successful due to the fact that radiation can induce a detrimental effect on biosensor reagent compositions. This detrimental effect can alter the biosensor's chemistry resulting in an inaccurate response during use.

Ideally, biosensor-based medical devices should be sterilized as an assembled and packaged product. Otherwise, a less economic approach of sterilizing individual components of the biosensor-based medical device followed by assembly and packaging of the device under clean and sterile conditions would be necessary.

Still needed in the field, therefore, is a simple and inexpensive method for manufacturing a biosensor-based medical device that yields a biosensor-based medical device that is both sterile and accurately calibrated. In addition, the method should enable the sterilization of an assembled and packaged biosensor-based medical device.

### SUMMARY OF THE INVENTION

Embodiments according to the present invention include methods for manufacturing a biosensor-based medical device that yields a biosensor-based medical device that is both sterile and accurately calibrated. In addition, the method enables the sterilization of an assembled and packaged biosensor-based medical device.

A method for manufacturing a sterilized and calibrated biosensor-based medical device (e.g., an integrated biosensor and lancet medical device) according to one exemplary embodiment of the present invention includes sterilizing at least one biosensor-based medical device that includes a biosensor reagent composition. The biosensor reagent composition can include, for example, an analyte specific enzyme and a mediator. The sterilizing can be accomplished using, for example, a gamma radiation-based technique. Thereafter, the biosensor reagent composition of the sterilized biosensor-based medical device(s) is calibrated.

A method for manufacturing a sterilized and calibrated biosensor-based medical device according to another exemplary embodiment of the present invention includes first assembling and packaging a plurality of biosensor-based medical devices that include a biosensor reagent composition. The packaged biosensor-based medical devices are then sterilized, using a radiation-based sterilization technique, to create a plurality of sterilized, packaged biosensor-based medical devices. Thereafter, the sterilized and packaged biosensor-based medical devices are calibrated. The calibration can be accomplished, for example, using a statistical sample of the sterilized, packaged biosensor-based medical devices.

Processes according to exemplary embodiments of the present invention provide for the manufacturing of a sterile biosensor-based medical device in an inexpensive manner by avoiding costs associated with assembling previously sterilized biosensor-based medical device components in a clean/sterile environment. Furthermore, highly accurate biosensor-based medical devices result from performing the sterilization step prior to the calibration step.

### BRIEF DESCRIPTION OF DRAWINGS

A better understanding of the features and advantages of the present invention will be obtained by reference to the following detailed description that sets forth illustrative embodiments, in which the principles of the invention are utilized, and the accompanying drawings of which:

FIG. 1 is a perspective view of a biosensor-based medical device (i.e., an electrochemical biosensor-based medical device) that can be utilized in certain embodiments of present invention;

FIG. 2 is a perspective view of another biosensor-based medical device (i.e., a colorimetric/photometric biosensor-based medical device) that can be utilized in certain embodiments of the present invention;

FIG. 3 is a flow chart illustrating a sequence of steps in a process according to one exemplary embodiment of the present invention; and

FIG. 4 is a flow chart illustrating a sequence of steps in a process according to another exemplary embodiment of the present invention.

### DETAILED DESCRIPTION OF THE INVENTION

Processes according to exemplary embodiments of the present invention can be employed to manufacture a variety of sterilized and accurately calibrated biosensor-based medical devices, including, but not limited to, the integrated biosensor and lancet medical devices described in U.S. patent application Ser. No. 10/143,399, which is fully incorporated herein by reference.

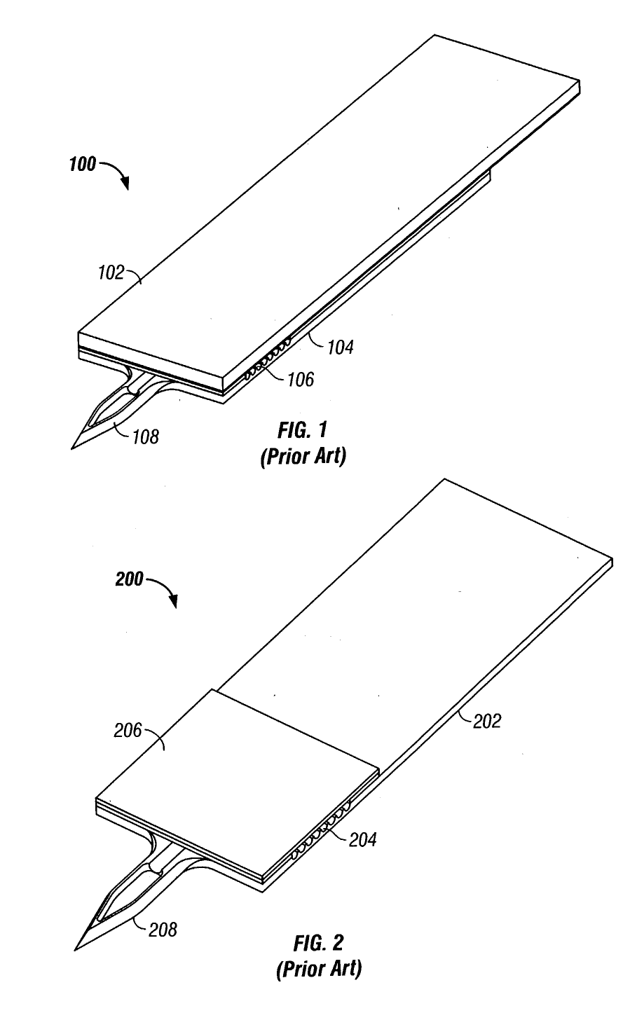

FIGS. 1 and 2 illustrate an electrochemical biosensor-based medical device and a colorimetric/photometric biosensor-based medical device, respectively, that can, for example, be manufactured by processes according to exemplary embodiments of the present invention.

Referring to FIG. 1, electrochemical biosensor-based medical device 100 includes a top electrode 102 and bottom electrode 104. The top electrode 102 and the bottom electrode 104 are held together by an adhesive layer (not shown). The adhesive layer is adapted to provide a reaction zone 106. Electrochemical biosensor-based medical device 100 also includes an integrated micro-needle 108 (also referred to as a lancet or an integrated lancet).

Furthermore, electrochemical biosensor-based medical device 100 includes a biosensor reagent composition (such as a redox reagent composition, not shown) present within reaction zone 106. The biosensor reagent composition is selected to interact with targeted component(s) (e.g., glucose) in a fluid sample (e.g., a whole blood sample) during an assay of the fluid sample. In electrochemical biosensor-based medical device 100, the biosensor reagent composition is disposed on top electrode 102 and resides within reaction zone 106.

In the configuration of FIG. 1, bottom electrode 104 is adapted to serve as a counter/reference electrode, while top electrode 102 is adapted to serve as a working electrode of an electrochemical cell. However, in other electrochemical biosensor-based medical device embodiments, and depending on a voltage sequence applied to the electrochemical cell, the role of the top and bottom electrodes can be reversed such that bottom electrode 104 serves as a working electrode, while top electrode 102 serves as a counter/reference electrode.

Suitable biosensor reagent compositions for electrochemical biosensor-based medical device 100 include, for example, an enzyme and a redox active component (e.g., a mediator). Further details related to electrochemical biosensor-based medical device 100 are discussed in U.S. Patent Application No. U.S. patent application Ser. No. 10/143,399.

FIG. 2 illustrates a colorimetric/photometric biosensor-based medical device 200 that includes a support substrate 202 made of an inert material, a matrix 204 for receiving a sample, a biosensor reagent composition (not illustrated) within matrix 204 that typically includes one or more members of an analyte oxidation signal producing system, and a top layer 206 (for example, a transparent top layer) which covers at least matrix 204. In other embodiments of a colorimetric/photometric biosensor-based medical device, top layer 206 can be, for example, a membrane containing a biosensor reagent composition impregnated therein, in which circumstance matrix 204 and the top layer 206 are mutually inclusive. Colorimetric/photometric biosensor-based medical device 200 also includes an integrated micro-needle 208 (also referred to as a lancet or an integrated lancet).

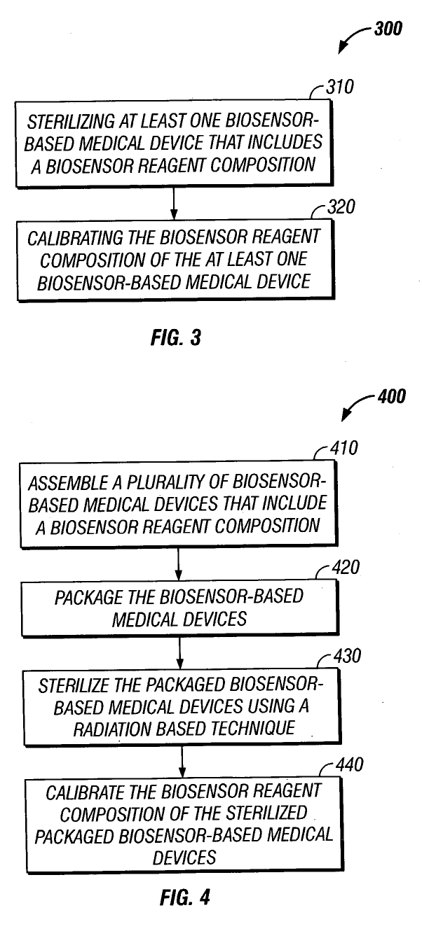

FIG. 3 is a flow chart illustrating a sequence of steps in a process 300 according to the present invention for manufacturing a sterilized and calibrated biosensor-based medical device. Process 300 includes the step of sterilizing at least one biosensor-based medical device (e.g., the medical devices of FIGS. 1 and 2 that include integrated lancets and biosensors, i.e., electrochemical and colorimetric/photometric sensors) to create at least one sterilized biosensor-based medical device, as set forth in step 310. The biosensor-based medical device(s) sterilized in step 310 includes a biosensor reagent composition.

Once apprised of the present disclosure, one skilled in the art will recognize that the present invention can be employed during the manufacturing of a variety of biosensor-based medical devices including, but not limited to, integrated biosensor and lancet devices described in U.S. patent application Ser. No. 10/143,399, which is hereby fully incorporated by reference.

Gamma sterilization can be considered appropriate for the complete destruction of harmful microbial flora in integrated biosensor and lancet devices that combine lancing, sample transfer and glucose concentration measuring (biosensor) components in a single integral disposable device. In such devices, a micro-needle is adapted to penetrate a subcutaneous skin layer, to access a blood sample and to transfer the blood sample to, for example, an electrochemical cell area of the device for glucose concentration determination. Therefore, the micro-needle must be provided in a sterile condition.

Process 300 is particularly beneficial for manufacturing a biosensor-based medical device that includes a biosensor reagent composition (e.g., a reagent composition that includes an analyte specific enzyme and associated mediator) whose analytical performance is altered upon exposure to radiation. For example, the analytical performance of a biosensor reagent composition that includes PQQ-based glucose dehydrogenase (a glucose specific enzyme) and ferricyanide (a mediator) has been determined as being altered by exposure to gamma radiation.

Sterilization step 310 can utilize any suitable sterilization technique. However, as will be described in detail below, processes according to exemplary embodiments of the present invention prove particularly useful when a radiation-based technique (e.g., a gamma radiation-based technique) is employed. Gamma radiation from a Co⁶⁰ source and a dose of 10 to 30 kGy can, for example, be used in sterilization step 310.

Next, the biosensor reagent composition of the at least one sterilized biosensor-based medical device is calibrated, as set forth in step 320. In order to avoid analytical inaccuracies resulting from changes in the analytical performance of a biosensor reagent composition due to sterilization step 310 (e.g., changes in calibration coefficients due to exposure of the biosensor reagent composition to gamma radiation), calibration step 320 is performed after sterilization step 310.

By performing calibration step 320 after sterilization step 310, effects of the sterilization step on the analytical performance of the biosensor-based medical device are compensated. For example, gamma radiation employed in a radiation-based sterilization technique can have an altering effect on the analytical performance of biosensor reagent compositions that include an analyte specific enzyme and a mediator. However, by conducting a calibration step subsequent to sterilization, such effects are compensated for during the calibration, thus providing an accurately calibrated biosensor-based medical device. This type of compensation can be particularly useful for integrated biosensor-based medical devices where a biosensor (e.g., an electrochemical cell biosensor or a colorimetric/photometric biosensor) and lancet are fabricated as a single integrated biosensor-based medical device.

FIG. 4 is a flow chart illustrating a sequence of steps in a process 400 according to the present invention for manufacturing a sterilized and calibrated biosensor-based medical device. Process 400 includes the step of assembling a plurality of biosensor-based medical devices, as set forth in step 410. The biosensor-based medical devices assembled in step 410 can be any suitable biosensor-based medical devices known to those skilled in the art. Process 400 is, however, particularly beneficial for manufacturing biosensor-based medical devices with a biosensor reagent composition and an integrated lancet, including those illustrated in FIGS. 1 and 2.

Assembly of the biosensor-based medical device can be accomplished using any suitable assembly technique known to those skilled in the art including, but not limited to, those described in U.S. patent application Ser. No. 10/143,399.

Next, at step 420, the biosensor-based medical devices assembled in step 410 are packaged to create packaged, biosensor-based medical devices. Such packaging encompasses, for example, cartridge form packages or individually wrapped devices in a card format package.

The packaged biosensor-based medical devices are then sterilized using a radiation-based sterilization technique, to create a plurality of sterilized, packaged biosensor-based medical devices, as set forth in step 430. In the circumstance that the biosensor-based medical devices include an integrated lancet, the sterilization step 430 is adapted to create a sterile lancet.

Next, the biosensor reagent composition of the sterilized, packaged biosensor-based medical devices are calibrated, as set forth in step 440. Only a fraction of a biosensor reagent composition batch used to assemble the plurality of biosensor-based medical devices need be used for the calibration step. For example, a sample (e.g., a statistically selected sample) of the sterilized, packaged biosensor-based medical devices can be calibrated versus a reference method. In this manner, calibration information (e.g., calibration coefficients) can be economically obtained for the remaining devices that were not part of the sample. In addition, calibration step 440 does not necessarily require clean/sterile room conditions, thereby not unduly increasing manufacturing cost.

Process 400 creates a sterile biosensor-based medical device in an inexpensive manner by avoiding costs associated with assembling previously sterilized components of a biosensor-based medical device (e.g., a previously sterilized lancet and an electrochemical test cell or photometric test strip) in a clean/sterile room. Furthermore, by performing sterilization prior to calibration, a highly accurate biosensor-based medical device is rendered.

In both process 300 and process 400, a sterilization step precedes a calibration step. This particular sequential order of steps (i.e., a sterilization step prior to a calibration step) enables the manufacturing of a sterilized and calibrated biosensor-based medical device of high accuracy and range, as demonstrated by Examples 1 and 2 below.

### EXAMPLE 1

### Effect of Gamma Radiation on the Enzyme Activity of a Biosensor Reagent Composition

Palladium (Pd) sputtered polyester panels (available from CP Films, Canoga Park, Calif.) were coated with a glucose sensitive biosensor reagent composition containing pyrroloquinoline quinone-glucose dehydrogenase (PQQ-GDH), pyrroloquinoline quinone (PQQ), potassium ferricyanide, a buffer and other components as set forth in Table 1 below. This biosensor reagent composition is described further in U.S. patent application Ser. No. 10/242,951, which is hereby fully incorporated by reference.

TABLE 1
Biosensor Reagent Composition

| Component | Weight (g) in 100 mL | % solids |
|---|---|---|
| Buffer (citraconate 66.7 mM): | 0.0273 | 0.0869 |
| Citraconic acid | | |
| Buffer (buffer pH 6.8): Dipotassium | 1.334 | 4.247 |
| Citraconate | | |
| Wetting agent (0.066%): Pluronic | 0.067 | 0.213 |
| P103 | | |
| Detergent (0.0332%): Pluronic F87 | 0.033 | 0.105 |
| Enzyme stabilizer (1.7 mM): CaCl₂ | 0.019 | 0.0605 |
| Stabilizer (75 mM): Sucrose | 2.5673 | 8.174 |
| Enzyme Cofactor (484 μM): PQQ | 0.016 | 0.051 |
| Enzyme (240 μM): PQQ-GDH | 2.647 | 8.428 |
| Mediator (750 mM): Potassium | 24.697 | 78.635 |
| Ferricyanide | | |
| Total solids: | 31.407 | 100.000 |

Dried Pd panels (size 6″ by 1.5″) coated with the biosensor reagent composition of Table 1 were packaged in KAPAK (Minneapolis, Minn.) pouches (1 panel per pouch) with silica gel desiccant and sealed under argon (Ar). The pouched samples were shipped to a sterilization facility together with a pouched control sample (i.e., a panel packaged in KPAK but that was not to be irradiated). A Gammacell 220 (serial no. 254) was used to irradiate (i.e., sterilize using a radiation-based technique) the samples. For this purpose, Co⁶⁰ was used as a source of gamma radiation. Sterilization was performed at Johnson & Johnson Sterilization Sciences & Technology (New Brunswick, N.J.).

Following sterilization with 10, 20 and 30 kGy doses of gamma radiation (without opening the pouches), the samples were returned and the PQQ-GDH activity assayed using the DCIP/PES (DCIP=2,6-Dichlorophenolindophenol Sodium salt, PES=phenazine ethosulfate) spectrophotometric method disclosed in U.S. patent application Ser. No. 10/242,951.

The 10, 20 and 30 kGy doses where chosen based on a belief that a 25 kGy dose of gamma radiation is commonly used in medical device industry. It was assumed, therefore, that a 25 kGy dose would be sufficient to produce a suitably sterile biosensor-based medical device, however no analysis of microorganism concentration following the radiation-based sterilization was conducted. Once apprised of the present disclosure, suitable radiation doses for use in processes according to the present invention can be readily determined by one skilled in the art without undue experimentation.

A Pd panel sample freshly coated with the biosensor reagent composition of Table 1 was prepared. Table 2 below shows the effect of the dose of gamma radiation on the activity of PQQ-GDH enzyme for each of the samples described above.

TABLE 2
Effect of gamma radiation on activity of the
PQQ-GDH enzyme coated Palladium Panel samples.

| | Radiation | Recovered | | % Change |
|---|---|---|---|---|
| | Exposure | Enzyme | Coefficient of | from |
| | Time | Activity | Variation % | radiation free |
| Sample Type | (min.) | (U/mL) | (n = 6) | sample |
| Fresh sample | N/A | 23.6 | 3.4 | N/A |
| Control sample | N/A | 24.1 | 1.5 | N/A |
| (not irradiated | | | | |
| but shipped to | | | | |
| and from the | | | | |
| sterilization | | | | |
| facility) | | | | |
| 10 kGy | 48.9 | 21.0 | 1.7 | −12.9 |
| 20 kGy | 97.8 | 21.9 | 1.1 | −9.1 |
| 30 kGy | 146.7 | 20.6 | 2.0 | −14.5 |

The data of Table 2 indicate a degradation of the biosensor reagent composition's enzyme activity following gamma radiation in comparison to samples that were not subjected to gamma radiation. If desired, such an activity degradation (loss of activity) can be inexpensively compensated by depositing a reagent composition with an enzyme activity that is higher in proportion to the expected loss due to gamma radiation sterilization. For example, for a 30 kGy gamma radiation dose, a reagent composition with a 15% higher enzyme activity could be employed to compensate for the expected 14.5% enzyme activity loss.

### EXAMPLE 2

### Effect of Calibrating Biosensor-based Medical Devices Before and After a Sterilization Step

Fully assembled and ready-for-use glucose biosensor-based medical devices including the reagent composition of Table 1 and gold and palladium electrodes located in an opposed configuration were obtained. Prior to gamma radiation sterilization, these devices were calibrated by testing with blood samples containing plasma equivalent glucose concentrations of 30, 270 and 620 mg/dL, as measured by a reference-instrument method using a standard YSI instrument (commercially available from Yellow Springs, Ohio). The calibration tests included blood samples With low, normal and high hematocrit levels (i.e., 20%, 42% and 70% hematocrit levels, respectively).

The biosensor reagent composition calibration step relies on collecting the response of multiple devices to blood samples of known plasma glucose concentration over a desired dynamic range (e.g., 20-600 mg/dL) and correlating the response to a reference method by minimizing differences between the two glucose readings. Ideally, the bias between the blood glucose concentration obtained from the biosensor-based medical device and from the glucose reference method for all blood samples should be zero. However, depending on glucose concentration and blood hematocrit, the bias can be non-zero (for example, up to ±15%). Typically, the following equation is obtained once a batch of biosensor-based medical devices have been calibrated:

Glucose_{YSI}=(Glucoseₛₑₙₛₒᵣ)^{a}+b

where:

“Glucose_{YSI}” is the glucose concentration as determined by the YSI reference instrument;

“Glucoseₛₑₙₛₒᵣ”=glucose concentration as determined by a biosensor-based medical device;

“a”=a coefficient which brings sensor response in-line with glucose concentration determined by the reference method; and

“b”=an offset (intercept) coefficient (observed, for example, when a glucose free blood sample is tested); the “b” coefficient an be either a positive or a negative number.

The calibration step described above rendered the following values of coefficients: a=0.6921 and b=0.5854, when performed prior to a sterilization step. Calibrated biosensor-based medical devices were packaged into KAPAK pouches containing silica gel desiccant, sealed and divided into four groups: (i) stored in the package at a controlled temperature and humidity environment (i.e., 20-25° C. and <10% relative humidity), (ii) shipment control, (iii) sterilized with 20 kGy dose, and (iv) sterilized with 25 kGy dose.

The last three groups of biosensor-based medical devices (i.e., groups [ii]-[iv]) were shipped to the same sterilization facility as in Example 1. Following radiation exposure, a blood glucose test was performed according to the same protocol as in the calibration step, using the a and b coefficients derived from the calibration step performed before sensor sterilization. Table 3 shows the averaged response of biosensor-based medical devices tested with 20, 42 and 70% hematocrit blood at three glucose concentrations (YSI values) and the bias of averaged response in mg/dL for the low glucose concentration or in % for the other two glucose concentrations.

TABLE 3
Response of glucose sensors sterilized at 20 and
25 kGy gamma radiation using calibration coefficients
obtained by performing a calibration prior to
sterilization (a = 0.6921, b = 0.5854); n = 18.

| | YSI | Avg. Sensor | Bias to YSI |
|---|---|---|---|
| Case | Glucose (mg/dL) | Glucose (mg/dL) | (mg/dL, or %) |
| 20 kGy | 32.7 | 44.5 | 11.8 |
| | 266.3 | 270.5 | 1.57 |
| | 606.0 | 565.8 | −6.64 |
| 25 kGy | 32.7 | 45.1 | 12.4 |
| | 266.3 | 268.3 | 0.73 |
| | 606.0 | 564.2 | −6.90 |
| Shipment Control | 32.7 | 28.1 | −4.56 |
| | 266.3 | 259.5 | −2.56 |
| | 606.0 | 571.0 | −5.77 |
| Stored in | 32.7 | 27.5 | −5.18 |
| Controlled | 266.3 | 264.6 | −0.65 |
| Environment | 606.0 | 571.1 | −5.76 |

The data of Table 3 indicate that, as an effect of sterilization using gamma radiation, a significant positive response bias at low glucose concentration is observed, rendering the biosensor-based medical devices relatively inaccurate at the glucose level where determination of hypoglycemia is critical to the patient treatment. On average, the YSI bias of devices irradiated at 20 and 25 kGy was about 12 mg/dL at the low (30 mg/dL) glucose concentration, whereas the bias of the shipping control and the sample stored in a controlled environment was only about −5 mg/dL.

Although no additional analysis has been performed, except for a measurement of the device background response, a conjecture based on the enzyme activity change reported in Example 1 is that the primary source of the increase in response bias is the formation of potassium ferrocyanide from the oxidized form of the mediator.

Next, the calibration procedure was performed following the gamma radiation process to demonstrate that a biosensor-based medical device of improved accuracy is obtained. Such a process sequence accounts for analytical performance changes resulting from interaction of the gamma rays with the biosensor reagent composition, thus delivering a biosensor reagent composition with an accurate response throughout the whole dynamic range of the system. Table 4 below contains the response of biosensor-based medical devices that were calibrated following the gamma radiation step.

TABLE 4
Response of glucose sensors irradiated at 20 and
25 kGy using calibration coefficients derived following
radiation sterilization. a = 0.7885, b = 1.088 for
the 20 kGy dosage; a = 0.7974, b = 1.1242 for
the 25 kGy dosage; n = 18.

| | YSI Glucose | Avg. Sensor | Bias to YSI |
|---|---|---|---|
| Case | (mg/dL) | Glucose (mg/dL) | (mg/dL, or % |
| 20 kGy | 32.7 | 32.9 | 0.18 |
| | 266.3 | 275.8 | 3.56 |
| | 606.0 | 601.0 | −0.82 |
| 25 kGy | 32.7 | 32.9 | 0.27 |
| | 266.3 | 274.4 | 3.02 |
| | 606.0 | 603.4 | −0.43 |

The results of Table 4 demonstrated a significant improvement in bias to YSI in comparison to Table 3, especially at the lowest glucose concentration. Thus, if the reagent calibration step is performed following radiation sterilization, the response bias to the reference method is minimized because the calibration parameters determined during calibration reflect (compensate) any changes in biosensor reagent chemistry.

It is speculated, without being bound, that gamma rays cause formation of ferrocyanide [Fe(CN)₆]⁻⁴ from the biosensor reagent composition mediator [Fe(CN)₆]⁻³. When a blood sample is tested on the biosensor-based medical device, an increase in reduced mediator concentration is interpreted by the device as additional glucose. In other words, gamma radiation of the biosensor-based medical device is speculated to affect enzyme activity and/or integrity of the mediator, generating quantities of product that are mistakenly detected as an analyte by the device, thus compromising the device's accuracy. However, if during manufacturing biosensor-based medical devices are irradiated first and calibrated following the sterilization step, the effect of radiation is compensated for rendering a highly accurate biosensor-based medical device.

Since a major response shift is observed in the intercept portion of the calibration following gamma radiation, the biosensor reagent composition can be calibrated in the last manufacturing step, thus avoiding costly clean room assembly procedures. In summary, when a sterilization step is performed prior to a calibration step, the bias seen in a process with the sequence reversed is not present.

It should be understood that various alternatives to the embodiments of the invention described herein may be employed in practicing the invention. It is intended that the following claims define the scope of the invention and that methods within the scope of these claims and their equivalents be covered thereby.

What is claimed is:

1. A method for manufacturing a sterilized and calibrated biosensor-based medical device, the method comprising:
sterilizing at least one biosensor-based medical device that includes a biosensor reagent composition, thereby creating at least one sterilized biosensor-based medical device; and
thereafter, calibrating the biosensor reagent composition of the at least one sterilized biosensor-based medical device.

2. The method of claim 1, wherein the sterilizing step utilizes a radiation-based sterilization technique.

3. The method of claim 2, wherein the sterilizing step utilizes a gamma radiation-based sterilization technique.

4. The method of claim 3, wherein the sterilizing step utilizes a gamma radiation dose in the range of 10 kGy to 30 kGy.

5. The method of claim 1, wherein the sterilizing step includes sterilizing a biosensor-based medical device with a biosensor based reagent composition that has an analyte specific enzyme and a mediator.

6. The method of claim 5, wherein the analyte specific enzyme includes PQQ and the mediator includes ferricyanide.

7. The method of claim 1, wherein the sterilizing step includes sterilizing a biosensor-based medical device comprising:
a biosensor reagent composition that includes:
an analyte specific enzyme; and
a mediator; and
an integrated lancet.

8. The method of claim 7, wherein the analyte specific enzyme includes PQQ and the mediator includes ferricyanide.

9. The method of claim 1 further comprising, prior to the sterilizing step, the step of:
packaging the at least one biosensor-based medical device.

10. The method of claim 1, wherein the biosensor-based medical device includes a reagent composition whose analytical performance is significantly altered upon exposure to radiation.

11. The method of claim 1, wherein the sterilizing step sterilizes a plurality of biosensor-based medical devices to create plurality of sterilized, biosensor-based medical devices and the sterilizing step utilizes a sample of the plurality of sterilized, biosensor-based medical devices.

12. A method for manufacturing a sterilized and calibrated biosensor-based medical device, the method comprising:
assembling a plurality of biosensor-based medical devices that include a biosensor reagent composition;
packaging the biosensor-based medical devices, thereby creating packaged biosensor-based medical devices;
sterilizing the packaged biosensor-based medical devices using a radiation-based sterilization technique, thereby creating a plurality of sterilized, packaged biosensor-based medical devices; and
thereafter, calibrating the biosensor reagent composition of the sterilized, packaged biosensor-based medical devices.

13. The method of claim 12, wherein the sterilizing step utilizes a gamma radiation dose in the range of 10 kGy to 30 kGy.

14. The method of claim 12, wherein the sterilizing step includes sterilizing a biosensor-based medical device with a biosensor reagent composition that includes an analyte specific enzyme and a mediator.

15. The method of claim 14, wherein the analyte specific enzyme includes PQQ and the mediator includes ferricyanide.

16. The method of claim 12, wherein the calibrating step utilizes a sample of the sterilized, packaged biosensor-based medical devices.

17. The method of claim 12, wherein the plurality of biosensor-based medical devices are a plurality of integrated biosensor and lancet medical devices.

18. The method of claim 17, wherein the integrated biosensor and lancet medical device is an electrochemical biosensor-based medical device.

19. The method of claim 17, wherein the integrated biosensor and lancet medical device is a photometric biosensor-based medical device.
